(19) United States
(12) Patent Application Publication
(10) Pub. No.: US 2014/0316521 A1
(43) Pub. Date: Oct. 23, 2014

(54) Rewriteable Aberration-Corrected Gradient-Index Intraocular Lenses

(71) Applicant: The Regents of the University of Colorado, a body corporate, Denver, CO (US)

(72) Inventors: Robert R. McLeod, Boulder, CO (US); Michael Cole, Longmont, CO (US)

(21) Appl. No.: 14/257,908

(22) Filed: Apr. 21, 2014

Related U.S. Application Data
(60) Provisional application No. 61/814,128, filed on Apr. 19, 2013.

(51) Int. Cl. A61F 2/16; G02C 7/02

(52) U.S. Cl. CPC A61F 2/1613; G02C 7/027; G02C 7/022
USPC 623/6.56; 351/159.74; 351/159.69

(57) ABSTRACT

Systems and methods for rewritable aberration-corrected gradient-index intraocular lenses are provided. Various embodiments relate to rewritable aberration-corrected gradient-index intraocular lenses. Some embodiments provide for polymer materials and processing to create full or partial rewritable phakic or pseudophakic intraocular lenses which allow for adjustable visual performance by doctors. Various methods to fabricate and adjust the lenses with optical and/or mechanical properties customized to the individual patient are also disclosed.

### CROSS-REFERENCE TO RELATED APPLICATIONS

This application claims priority to U.S. Provisional Patent Application No. 61/814,128 filed Apr. 19, 2013, which is incorporated herein by reference for all purposes.

### STATEMENT REGARDING FEDERALLY SPONSORED RESEARCH

This invention was made with government support under grant number IIPO822695 awarded by the National Science Foundation. The government has certain rights in the invention.

### TECHNICAL FIELD

Various embodiments of the present invention generally relate to systems and methods for creating customized lenses. In particular, some embodiments of the present invention relate to systems and methods for creating rewritable aberration-corrected gradient index lenses.

### BACKGROUND

A lens is an object that can be used to alter the behavior of light. For example, a lens can transmit and refract light towards a focal point. Lenses are typically made of plastic or glass and can be used in a wide range of applications and imaging systems. For example, lenses can be found in binoculars, telescopes, endoscopic probes, microscopes, projectors, cameras, and projectors all use lenses. In addition, corrective lenses such as eye glasses and contacts can be used for the correction of visual impairments (e.g., defocus, astigmatism, and higher-order aberrations).

Given the variety of applications and types of objectives, it has traditionally been impractical to stock all lenses that could possibly be needed. With corrective lenses, for example, the accuracy of the correction is limited by the number of lenses that can economically be manufactured and stocked. Thus adding finer divisions or higher order aberrations (e.g., coma) would improve patient vision but at the cost of much larger inventory, which becomes expensive to fabricate and maintain. In addition, traditional systems for creating customized lenses that correct for various aberrations are expensive and can have a significant lag time. As a result, systems and methods are needed for efficiently creating customized lenses.

### SUMMARY

Various embodiments include methods, systems, and devices that may be used to create, modify, and/or customize rewriteable aberration-corrected gradient index lenses (e.g., intraocular lenses (“IOLS”)) and other ophthalmic devices. In some embodiments, aberration data is received. The aberration data may correspond to measurements specific to a patient, specifications to correct a specific aberration (e.g., near-sightedness of 2 diopters), may indicate no aberrations at all (e.g., a person with perfect vision who none-the-less needs an intraocular lens), used to create a multiple foci or one or more foci whose shape has been designed to improve vision, (e.g., an extended depth of focus), and/or an arbitrary function which can be used to create particular aberrations. These aberration data or attributes may be written to the lens and eventually updated as needed through an erasure (or degradation) process and rewriting process. For example, an ophthalmic device may be an intraocular lens composed of a material capable of at least one write step and one degradation step. However, the materials may be capable of multiple writes and degradations. As another example, the ophthalmic device may be a torric intraocular lens and the aberration data may be recorded after insertion in an eye and after the lens has settled or stabilized its position in the eye.

In accordance with various embodiments, the material may include crosslinked polymeric material capable of recording patterned light as refractive index changes and/or a change of lens refractive power via shape or surface profile modification can be recorded onto the device using changes in refractive index. Using various techniques, the aberration data may be recorded, erased, and/or rerecorded including cases where the device is in the eye. In some cases, other data may be recorded to the lens. Examples include, but are not limited to, a patient's ophthalmic history, a patient's prescription history, a patient's identification information, device information, recording and/or erasing parameters. In addition to an erasure process, the material may allow a degradation step that also allows for physical alterations (temporary or permanent) of the lens or other ophthalmic device. In some embodiments, the degradation can allow for the facile removal of an intraocular lens. For example, the degradation step can result in a change in shape of the lens such that the lens curls up tightly to facilitate removal through a small incision.

Some embodiments provide for an ophthalmic device using a crosslinked polymeric material with freely diffusing species capable of bonding with the crosslinked polymeric material. The ophthalmic deice can be, but is not limited to, one or more of the following devices: a phakic lens, an intraocular lens, or a contact lens. In some embodiments, the device can be shaped by at least one of milling, lathing, and/or molding. In accordance with other embodiments, the device may be capable of changing its refraction and/or diffraction attributes by the use of photochemistry.

The material of the ophthalmic device may include materials that have reversible chemistries which use one or more of the following groups: anthracenes, acenaphthylenes, phenanthrenes, and/or related polyaromatic hydrocarbons, stilbenes, coumarins, maleimides, thymines, and uracils. In some embodiments, the reversible chemistry may use one or more of the following groups: spiropyrans, pirooxazines, and/or azobenzenes. The reversible chemistry may be reversible for more than 5 cycles with less than 25% loss of maximum change in refraction and/or diffraction attributes in various embodiments. In addition, the material may include UV blockers.

In some embodiments, an ophthalmic device can be created using a cross-linked polymeric material with no freely diffusing species contained within the device other than water or water and saline mixtures (e.g., aqueous humor of the eye) for use in one of the following devices: a phakic lens, an intraocular lens, or a contact lens. The device may be shaped by at least one or more of the following techniques: milling, lathing, and/or molding. In some embodiments, the device is capable of changing its refraction and/or diffraction attributes by the use of photochemistry; whereby the photochemistry changes the concentration of water over a portion or over the whole of the device. The refractive index contrast obtained during a writing step may be greater than 0.005 over 1 mm between an exposed region and an unexposed region. The device may be capable of water concentration changes greater than 5 wt % upon exposure to light to which the material is sensitive. The device may swell or shrink with volume changes greater than 10% upon exposure to light to which the material is sensitive.

The chemistry causing the change in water concentration may be from photocyclodimerization of the matrix with itself, using one or more of the following groups: anthracenes, acenaphthylenes, phenanthrenes, related polyaromatic hydrocarbons, stilbenes, coumarins, maleimides, thymines, and/or uracils. The chemistry causing the change in water concentration may be from at least one of the following: Spiropyrans, pirooxazines, azobenzenes. In some embodiments, the chemistry causing the change in water concentration is photoreversible. The change in water concentration may be reversible for greater than five cycles with less than 25% loss of maximum change in concentration. In some embodiments, the device may be an intraocular lens, and the changes in water concentration occur while the device is in the eye.

Some embodiments provide for an ophthalmic device that uses a crosslinked polymeric material capable of recording erasable refractive index patterns for more than ten write-erase cycles with less than 20% degradation to the maximum refractive index contrast of which the material is capable. The ophthalmic device may be optimized for use with other eyewear such as, but not limited to, traditional glasses or an exterior head mounted display. In some embodiments, the ophthalmic device may have surface features that were molded, milled, and/or lathed onto the device. In addition, the ophthalmic device may be comprised of a material capable of recording light patterns as refractive index patterns in the volume of the material. The refractive index patterns may be rewriteable.

In some embodiments, a previously recorded refractive index pattern recorded on the rewriteable ophthalmic device may be fully erased or partially erased. The refractive index pattern on the rewriteable ophthalmic device may include the placement of multiple focus zones, multiple foci, or focal zones with a particular shape including extension of the focal spot along the optic axis. In addition, some embodiments, track the trajectory of the patient's eye health and recording visual correction that gets better with time. As a result, various embodiments lengthen the time needed between visits. In some cases, the trajectory of the visual correction may move in any direction.

Embodiments of the present invention also include computer-readable storage media containing sets of instructions to cause one or more processors to perform the methods, variations of the methods, and other operations described herein.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

Embodiments of the present invention will be described and explained through the use of the accompanying drawings in which:

FIG. 1 is a flowchart illustrating a set of operations for recording a refractive index pattern on a rewriteable ophthalmic device in accordance with various embodiments of the present invention;

FIG. 2 is a flowchart illustrating a set of operations for adjusting a refractive index based on patent feedback according to some embodiments of the present invention;

FIG. 3 illustrates various operations for creating customized refractive index patterns on a rewriteable ophthalmic device in accordance with one or more embodiments of the present invention;

FIG. 4 is a block diagram illustrating various component which may be used in a systems, devices, components, or engines in accordance with at least one embodiment of the present invention; and

FIG. 5 illustrates an exemplary computer system that may be used in one or more embodiments of the present invention.

The drawings have not necessarily been drawn to scale. For example, the dimensions of some of the elements in the figures may be expanded or reduced to help improve the understanding of the embodiments of the present invention. Similarly, some components and/or operations may be separated into different blocks or combined into a single block for the purposes of discussion of some of the embodiments of the present invention. Moreover, while the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Previous polymeric materials that have been used in ophthalmic devices are typically solid materials with a single refractive index throughout the material. These materials rely on their shape to create a lens. More recently, more advance materials have been created that have gradient refractive index structures or even sharp changes in refractive index distributed spatially throughout the material. These newer materials have the advantage of greater numerical apertures and greater control over lens functions such as multiple foci, focal zones, or planes. Even more advanced are the materials that allow the eye specialist to record the spatially varying refractive index pattern into the material while it is in the eye. Such materials are well suited for intraocular lenses and phakic lenses whereby the material is surgically inserted into the eye, allowed to settle, and then the refractive index pattern needed to correct that patient's vision is recorded into the material. U.S. Patent Publication No. 2006/0271186, U.S. Pat. No. 6,450,642, and U.S. Patent Publication No. 2009/0287306 provide more details and are hereby incorporated by reference in their entirety for all purposes.

However, despite the advanced state of the current technologies, they still have a disadvantage that prevents these materials and the technology from gaining larger acceptance, and that is that the patient's vision is seldom stable over the course of years. Since the materials are typically surgically inserted into the eye, the prospect of needing surgery again in 5 to 10 years severely limits the adoption of the technology by both doctors and patients. Even Lasik eye surgery has a similar limitation in that the surgery can be done only once in many cases and up to three times in the best cases. Therefore, many patients with moderately to rapidly changing prescriptions will opt for regular glasses, and those that opt for surgery face the possibility of surgery again in the future and all the cost and discomfort that accompanies it. In some cases, the post-surgical performance may not meet patient desires and it may be desirable to modify the lens power, aberrations such as astigmatism, number of focal zones, multiple foci, or other properties several times without resorting to additional surgery.

Various embodiments of the present invention solve the multiple surgery issue, as well as the limitations of Lasik, by offering a polymeric material for ophthalmic devices that is rewritable (fully or partially). With this new material, the ophthalmic device can be surgically inserted into the eye, allowed to settle, and then record a spatially varying refractive index pattern just like the current advanced materials and procedures. The difference is that the refractive index pattern can be erased and a different refractive index pattern can be recorded into the material at any time. Thus, even years later, a new prescription for vision correction can be recorded into the material without the need for surgery. Some examples of, but not limited to, devices that can use this material are contact lenses, phakic or pseudophakic intraocular lenses, and intraocular lenses.

In accordance with various embodiments, the focusing power of a lens can be created in several ways. For example, the surface of the lens can be curved to bend the light as it refracts through the surface. The surface can also be structured into the form of a diffraction grating such that light is bent and possibly split at the surface. The refractive index of the body of the lens can be non-uniform such that light is bent in response to the gradient of this refractive index variation. In addition, the refractive index and/or absorption of the body of the lens can be modulated into a diffraction grating such that the light is bent and possibly split within the body. These methods can be combined in various ways to achieve a desired property such as compensation of chromatic variations of the lens function, splitting the light into multiple foci or controlling the response of the lens to color and/or angle of the incident light. Generally, the function of the lens implemented by these methods or others is referred to as the prescription of the lens.

While, for convenience, embodiments of the present invention are described with reference to creating customized lenses, embodiments of the present invention are equally applicable to various other types of optical devices including, but not limited to, holograms, diffraction gratings, optical waveguides, creating optical functionality to support other devices (e.g., cameras) embedded within an intraocular lens or other ophthalmic device. In addition, in the following description and attached appendices, for the purposes of explanation, numerous specific details are set forth in order to provide a thorough understanding of embodiments of the present invention. It will be apparent, however, to one skilled in the art that embodiments of the present invention may be practiced without some of these specific details.

Embodiments of the present invention may be provided as a computer program product which may include a machine-readable medium having stored thereon instructions which may be used to program a computer (or other devices or machines) to perform a process or to cause a process to be performed. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments of the present invention may also be downloaded as a computer program product, wherein the program may be transferred from a remote computer to a requesting computer by way of data signals embodied in a carrier wave or other propagation medium via a communication link (e.g., a modem or network connection).

### Terminology

Brief definitions of terms used throughout this application and attached Appendix are given below.

The terms “connected” or “coupled” and related terms are used in an operational sense and are not necessarily limited to a direct connection or coupling.

The term “embodiments,” phrases such as “in one embodiment,” and the like, generally mean the particular feature(s), structure(s), method(s), or characteristic(s) following or preceding the term or phrase is included in at least one embodiment of the present invention, and may be included in more than one embodiment of the present invention. In addition, such terms or phrases do not necessarily refer to the same embodiments.

If the specification states a component or feature “may”, “can”, “could”, or “might” be included or have a characteristic, that particular component or feature is not required to be included or have the characteristic.

The term “module” refers broadly to a software, hardware, or firmware (or any combination thereof) component. Modules are typically functional components that can generate useful data or other output using specified input(s). A module may or may not be self-contained. An application program (also called an “application”) may include one or more modules, and/or a module can include one or more application programs.

### General Description

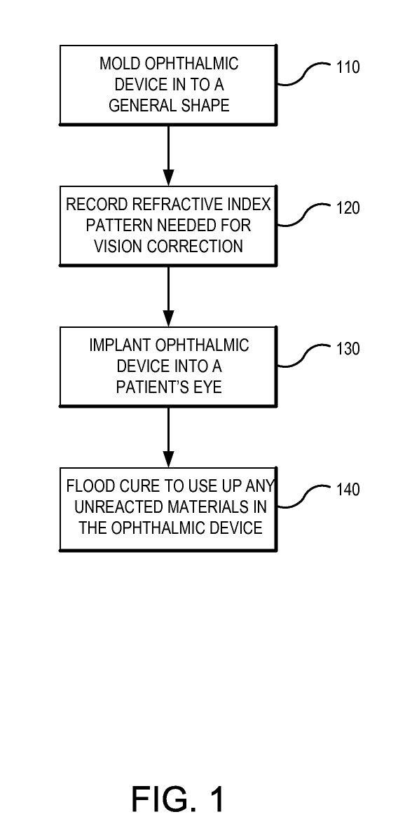

The process for making ophthalmic devices can be quite varied, but typically follows a general outline as illustrated in FIG. 1. First, during molding operation 110, the material that is to become the ophthalmic device is molded into the general shape (referred to a blank) that it needs for use in the eye. Then, during recording operation 120, the refractive index pattern or prescription needed for vision correction is recorded into the ophthalmic device. The ophthalmic device is then placed in the eye during insertion operation 130. For the more advanced materials and procedures, the blank is inserted into the eye, allowed to settle or is pinned to the eye, and then the refractive index pattern or prescription is recorded into the ophthalmic device. One or more optical exposures (e.g., flood cure) of the device may then be used to use up any unreacted materials in the device during curing operation 140. The optical exposures make the device stable against further changes in refractive index should the device be exposed to wavelengths of light used to record the refractive index pattern. The type and number of optical exposures selected may depend on chemistries, desired outcome, and other factors.

For various embodiments of the present invention, whether in the eye or not, the potential to correct any mistakes that occurred during recording process or even add to the recorded pattern with overlapping recordings that further refine the ability of the device to correct the patient's vision. For more details on the processing of ophthalmic devices and the recording process can be found in U.S. patent application Ser. No. 13/715,606 entitled “Systems And Methods For Creating Aberration-Corrected Gradient Index Lenses” filed on Dec. 14, 2012, U.S. patent application Ser. No. 13/849,256 entitled “Liquid Deposition Photolithography” filed on Mar. 22, 2013, U.S. Patent Publication No. 2006/0271186, U.S. Pat. No. 6,450,642, U.S. Patent Publication No. 2009/0287306, U.S. Patent Publication No. 2013/0268072, U.S. Pat. No. 5,147,394, and U.S. Pat. No. 8,292,952, all of which are hereby incorporated by reference in their entirety for all purposes.

The material used in various embodiments can best be described by having two major components: 1) a matrix component; and 2) a writing component. The matrix component can be a polymeric material that is typically of low refractive index. The polymeric material can be organic, inorganic, or hybrid organic-inorganic polymer. Some examples of materials that can be used in such devices can be found in the previously listed US patents and applications. The following additional references provide examples of the types of materials that are useful and are all hereby incorporated in their entirety for all purposes: U.S. Pat. No. 6,939,648, U.S. Pat. No. 6,103,434, U.S. Pat. No. 8,071,260, U.S. Pat. No. 7,521,154, and U.S. Pat. No. 8,062,809. The main function of the matrix is to provide support and structure to the ophthalmic device. Its bulk modulus range is preferably between (2.2 GPa) to (35 GPa) and more preferably on the lower end of the scale. In a few embodiments, the bulk modulus may fall outside the range given and are also acceptable.

The lens material can have a glass transition temperature (Tg) that can vary from −100° C. to 200° C. However, for most embodiments, the lens material (either before or after optical patterning has occurred, and before or after insertion into the eye), will be less than 40° C. during the optical patterning of the material. The low Tg during recording allows for facile diffusion of the writing component in the matrix component. After recording or exposure to an optical pattern, there may be the option of increasing the modulus and/or the Tg with either photoreaction or wet chemistry. Diffusion of the writing component is important for development of refractive index structures. Lower Tgs typically translates into faster diffusion rates for small molecules that are dissolved in the matrix material. Faster diffusion rates translate into shorter recording time. Ideal recording times are less than 1 minute per exposure, and more preferably less than 1 second, and most preferably less than 1 millisecond. Most hydrogels (used in contact lenses as well as many hydrophilic IOLS (and phakics), have very fast diffusion characteristics due to the water inside the hydrogel. Materials that are not hydrophilic but composed of silicones can also have fast diffusion characteristics for the writing component as long as the Tg is low as described. When needed, heat can be applied and/or solvents can be added to increase diffusion rates.

A session for changing the refractive index of the lens may involve one or more exposures to create the desired refractive index profile on the lens, thus a session desirably should take less than 15 minutes, and more preferably less than 5 minutes, and most preferably less than 1 minute. After a session, the results of the correction may take several days to reach equilibrium (based on diffusion times). The fixing of the chemistry may be immediate in some sessions, whereas in others, fixing the chemistry may wait until all materials have diffused to an equilibrium state which is some cases may take a month but preferably takes less than one week, and more preferably less than one day, and most preferably less than one hour. Fixing the chemistry (or material or media) refers to the process of locking down any of the diffusible chemistry present in the media and this can be done by leaching the material out using a solvent bath or by wet chemistry methods to react with the diffusing species such that they become nondiffusing, or more preferably by using light of a wavelength that causes the diffusible species to become nondiffusing by whatever mechanism triggered by the light. In some embodiments, no fixing is necessary.

The light intensity used to record the refractive index patterns may be dependent on a number of factors (i.e., wavelength, dose, material qualities, etc.). Each of the various embodiments may require a different light dosing requirement. For instance, if multiphoton irradiation is being used to create changes inside the device, then very large powers or intensities may be used. If single photon reactions are being used to create the changes inside the device, then lower intensities will likely be used. To select the right light conditions, the factors to consider are the reactivity of the material (a function of the reactive groups, the concentration of the groups, and the efficiency of the reactions taking place), the absorbance of the material, the intensity of the light, the wavelength(s) used, and the amount of time the light irradiates the sample. It is usually best to find the best conditions for each material in a laboratory setting. In cases whereby the refractive index, the shape, and/or the surface features are being modified outside of the eye, standard laboratory or manufacturing conditions and light sources can be used. However, when the device is being modified with light while in/on the eye, lab research and modeling can determine the safe intensities, powers, and wavelengths that can be used in/on the eye.

The wavelength used to record the light intensity pattern into the blank can be any wavelength from 700 nm to 180 nm. Preferably, the refractive index pattern is recorded from 410 nm to 250 nm. When the material is photo-erasable, the erasing of the refractive index pattern is performed at wavelengths from 700 nm to 150 nm, more preferably from 400 nm to 180 nm, and most preferably from 200 nm to 370 nm. In other embodiments, mutliphoton techniques may be used that include intense, short pulses typically in the 500 nm to 800 nm range (though wavelengths outside this range are also possible). The single or multiphoton wavelength can be chosen by measuring the absorption spectrum of the chemistry that is being used in the material and then matching a light source to that wavelength. Lasers are usually preferred and may be pulsed or continuous output. LEDs, fluorescent, mercury lamps, flash lamps, and other light sources are also possible.

In one embodiment, the matrix can be formed into the desired ophthalmic device shape before insertion into the eye using methods already known in the art (use of a mold and cure matrix in the mold to form a blank, cold milled, heat molded, injection molded, lathed, and/or other methods). The matrix precursors can form the matrix by any number of different methods such as free radical polymerization, Diels-Alder chemistry, ionic polymerization, ring opening polymerization, thiolene chemistry, Michael additions, silicone-hydride polymerizations, silicone hydrolysis, sol-gel reactions, water catalyzed polymerizations and many more. The matrix may also be formed from condensation types of chemistry such as isocyanate-hydroxyl, carboxylic acid-amine, acid chloride-amine, isocyanate-thiol, ester-amine, ketone-amine, aldehyde-hydroxyl, and many more. The above describes some of the reactive chemistries that can be used to form a preferably crosslinked matrix. In some embodiments, the matrix may not be crosslinked but rather be oligomers or thermoplastic polymers, but crosslinked matrices are preferred in some embodiments. The bulk of the matrix typically will consist of chemical moieties that are different from the reactive chemistry used to form the matrix. For instance, if polyethylene glycol (PEG) acrylates were used to form the matrix then the matrix would consist primarily of the PEG. As another example, if the reactive groups were isocyanate-hydroxyl, whereby the isocyanate was isophorone diisocyanate and the hydroxyl was polydimethyl siloxane with carbinol (also known as hydroxyl) reactive groups, then the bulk of the material would have cycloaliphatic groups, dimethylsiloxane groups, and urethane groups and no longer contain significant amounts isocyanate or hydroxyl. However, some embodiments that use reactive groups that facilitate hydrophilicity such as hydroxyl groups (like the hydroxyl-isocyanate example above) may be formulated to be in large excess such that all isocyanate groups are reacted and leave hydroxyl groups in significant amounts.

In embodiments whereby the blank is formed in a mold, the matrix for the lens can be formed in a blank from a matrix precursor by a curing step (curing can be thermally and includes room temperature cures), with light irradiation (using a mold with transparency at the wavelength needed to initiate reaction of the matrix precursors), or injection of a charged particle catalyst (i.e., alpha or beta radiation)). It is possible for the matrix precursor to be one or more monomers, one or more oligomers, or a mixture of monomer and oligomer. The matrix precursor can even be a thermoplastic polymer in some embodiments, in which case, telechelic polymers are preferred. In addition, it is possible for there to be greater than one type of precursor functional group, either on a single precursor molecule or in a group of precursor molecules. Precursor functional groups are the group or groups on a precursor molecule that are the reaction sites for polymerization during the matrix cure. The precursor is advantageously liquid at room temperatures, but some heating to form a liquid is acceptable (such as with thermoplastic precursors or low melting oligomers). The curing of the matrix to form a blank should preferably take less than five minutes, more preferably less than a minute, most preferably, less than 10 seconds. In embodiments whereby the lens is formed from milling, lathing, thermal molding, or vacuum molding, the matrix precursors are typically thermoplastic or even thermosets. The making of a thermoset for milling or lathing or similar processes can be identical to that as described for matrix precursors reacted in molds as described previously. In some embodiments where thermoplastics are used, it may be advantageous that the material become crosslinked during the patterning stage with light. In yet other embodiments, hybrid manufacturing methods may be employed in which two or more of the above methods for forming a blank are used. For example, a blank may be formed via reaction injection molding (RIM), cured thermally, then lathed, then milled.

When the matrix precursor is polymerized using any of the said functional groups, a number of different catalyst can be used and are selected depending on the polymerization reaction occurring. For example, cationic epoxy polymerization takes place rapidly at room temperature by use of BF3-based catalysts, other cationic polymerizations proceed in the presence of protons, epoxy-mercaptan reactions and Michael additions are accelerated by bases such as amines, hydrosilylation proceeds rapidly in the presence of transition metal catalysts such as platinum, peroxides or other thermal radical generators are useful for acrylate and methacrylate cures, and urethane and urea formation proceed rapidly when tin or bismuth catalysts are employed. Photoinitiators can also be used to cure the matrix. Some typical photoinitiators are acylphosphine oxides, titanocene derivatives, and various acetophenone derivatives.

In some cases, curing of the matrix does not affect or interfere with the writing chemistry. The two processes (curing of the matrix and the writing chemistry) should be selected such that they are chemically orthogonal. An example of orthogonal chemistry is isocyanate-hydroxyl (to form polyurethane) as the matrix forming chemistry and photodimerization of anthracenes as the writing chemistry. However, in a few embodiments, cross reaction is inevitable and can even be useful. In such cases, up to 50% incorporation of the writing component can be acceptable, though lower percentages may be preferred with less than 15% incorporation being the most preferred. An example of this latter case would be the use of an acrylate-vinyl ether free radical cure with excess vinyl ether functionality to create a crosslinked matrix with pendant vinyl ether functionality and then use cyanoacenapthylene as a freely diffusing writing component (photodimers with vinyl ether reversibly); many of the vinyl ethers will be copolymerized with the acrylate to help form the low index matrix and to prevent any incorporation of the cyanoacenapthylene, it can be pre-dimerized with vinyl ether functional groups.

In embodiments whereby the writing chemistry used to form refractive index patterns, hydrophylicity changes, and/or volume changes is freely diffusing, a feature of the matrix is that the matrix has the ability to capture the writing component. The ability of the matrix to capture writing components is from functional groups on the matrix with which the writing components photo-react. These reactive groups can be a part of the matrix backbone or pendant to the matrix backbone. The reactive groups are the same types of reactive groups described for the writing components; the reactive group may be any group that allows attachment to either other writing components and/or to the matrix. For example, reactive groups that are capable of photodimerization, photoinsertion, photo-diels alder reactions are preferred, more preferred reversible reactions, most preferred are photoreversible reactions as exemplified by 2+2, 4+4 photocyclization reactions. The matrix reactive groups may all be the same or may be different mixtures of matrix reactive groups (for example, all same=all vinyl ethers, mixture=vinyl ethers and coumarins). Thus, the matrix reactive groups comprises at least one type of reactive group and these matrix reactive groups may be the same or different from the freely diffusing writing component in embodiments that use a freely diffusing writing component.

The writing component typically has a high refractive index group and a reactive group. The high refractive index groups will typically contain one or more aromatic rings, heavy atoms (bromine, iodine, sulfur, bismuth, etc.), and polarizable atom systems (conjugated systems). The reactive group is any group that allows attachment to either other writing components and/or to the matrix. For example, reactive groups that are capable of photodimerization, photoinsertion, photo-diels alder reactions are preferred, more preferred reversible reactions, most preferred are photoreversible reactions as exemplified by 2+2, 4+4 photocyclization reactions.

While refractive index is one characteristic that can be modified during the photoreaction associated with the writing step, other characteristics can also be modified with the writing step photoreaction of freely diffusing writing components such as hydrophilicity and shape. The writing component may also be used to modify the hydrophilicity of a region on/in the material. To bring about a change in hydrophilicity, very polar groups such as hydroxyl, sulfones, carboxy acids, sulfur based acids, phosphorous based acids, amides, ketones, amines, and salts can be used. Also, a change in hydrophilicity can be accomplished by use of nonpolar groups such as alkyl siloxanes, fluorinated molecules, and alkanes just to list a few. The change in hydrophilicity can bring about a change in shape, either on the surface of the device and/or in the volume of the device; and though the preferred change in shape and/or volume is caused by the movement of water, other polar molecules (such as amides, glycerin derivatives, salts, acids, etc.) can be used to diffuse into or out of a regions whose hydrophilicity has changed. The same is true for the converse case whereby nonpolar molecules are diffusing into or out of a region whose hydrophilicity has changed.

The writing component may be monofunctional or multifunctional and the reactive groups may be the same type or different types and may be on the same molecule or on different molecules (i.e., an acrylate group and an acenapthylene group on the same molecule represents two different types of reactive groups on the same molecule, where as a mixture of acrylates and acenaphthylenes as separate molecules is another example). Additionally, the reactive groups may be the same or different from the reactive groups present on the matrix for binding the writing component. The writing components themselves may be organic, inorganic and organic/inorganic hybrids.

The ratio of reactive matrix groups on the matrix versus freely diffusing writing components can be 1/10, more preferably the ratio is greater than 1/1, most preferably the ratio is greater than 10/1. A larger concentration of binding groups on the matrix relative to the writing components insures that the matrix does not become saturated with bound writing components in a given location which would limit the contrast potential for low refractive index regions compared to high refractive index regions. In some embodiments, the writing component is multifunctional and is capable of binding with itself and with the matrix reactive groups, which means that the matrix reactive binding sites do not need to be as concentrated since saturation of matrix binding sites is not lost.

Suitable write components include molecules containing C—C double bonds that undergo any of the various types of reversible photocycloaddition reactions. These can include anthracenes, acenaphthylenes, phenanthrenes, related polyaromatic hydrocarbons, stilbenes, coumarins, maleimides, photodiene formation/Diels Alder reaction, and concerted and nonconcerted ene-ene reactions (2+2, 4+4, 4+2, 3+2, etc.). Of particular interest are uracil and thymine and similar natural compounds that undergo photo-cyclodimerization. Acenapthylenes are also of particular interest including the reaction of electron withdrawn acenaphtylenes (i.e., cyanoacenapthylene) with itself or with vinyl ethers). Also, metal and organic salts can be attached to photochelating groups, such as spiro compounds (such as various spiropyrans and spirooxazines), chromenes, and the like. Nucleotides, such as DNA and RNA, can also be attached to such photochelating compounds via strong hydrogen bonding interactions.

Polymer bound metal complexes can be used as reactive sites for photoinsertion or photoexchange of various ligands. Molecules used as photoinitiators for polymerization can attach to the matrix via reactive groups such as C—C double bonds. Thiols, selenols, tellenols, disulfides, diselenides, ditellurides, and various photoiniferters can also bind to the matrix via reactive sites composed of C—C double bonds (other types of unsaturation such as heteroatomic enes or ynes are also contemplated). For best results, the high refractive index moiety should be chosen as part of the writing component and when possible, a lower refractive index component is part of the matrix reactive group. Of course, such role distinctions can be reversed in some embodiments such that high refractive index components may be a part of the matrix and low refractive index components for writing. Other reactive chemistries that are not listed are also considered and thus the list above should not be considered all inclusive. This list should in no way be construed as complete. Preferably, the reactive chemistry used for binding the write components to themselves and/or to the matrix should be reversible. Any of the chemistry described for the write components can be part of the matrix reactive groups. In some embodiments, the writing chemistry is not freely diffusing and is wholly part of the matrix.

The reversibility can be from any number of different processes. For instance, the binding of the writing component to the matrix may occur via a thermal reaction and then be released by a photoreaction whereby the process can then be repeated. Preferable reversible reactions consist of photobinding of writing component (to itself and/or to the matrix) and photorelease of the writing component, whereby the binding and release cycle can be performed more than once.

There are at least three mechanisms for changing the refractive index of a material used in various embodiments. The following are examples of increasing the refractive index of the material or a portion of the material of the device. One, an increase in refractive index can be accomplished by the diffusion and binding of high refractive index molecules into a region of the material. Second, the refractive index can be increased by densification of the material of the present invention. Thirdly, the refractive index of the material can be increased by the outward diffusion of a low refractive index molecule (such as water). The outward (or inward) diffusion of the low refractive index molecule can be controlled by changes in solubility of the local region towards that molecule. It is understood that increases or decreases in the refractive index are possible and useful in various embodiments. In many embodiments, these mechanisms for change in refractive index may be reversible. These mechanisms may also swell or shrink the material, resulting in a change of optical function which is a combination of the refractive index change and a shape/volume change.

The first mechanism using binding of molecules to the matrix is described in more detail in U.S. Pat. No. 7,521,154 and U.S. Pat. No. 8,062,809, which are hereby incorporated by reference in their entirety for all purposes. This first mechanism is also described in radical polymerization of monomers and oligomers either inside a polymeric matrix or to form a polymeric matrix. The following documents describe such processes: U.S. patent application Ser. No. 13/715,606 entitled “Systems And Methods For Creating Aberration-Corrected Gradient Index Lenses” filed on Dec. 14, 2012, U.S. patent application Ser. No. 13/849,256 entitled “Liquid Deposition Photolithography” filed on Mar. 22, 2013, U.S. Patent Publication No. 2006/0271186, U.S. Pat. No. 6,450,642, and U.S. Publication No. 2009/0287306, all of which are hereby incorporated by reference in their entirety for all purposes. The second mechanism uses reactive groups on the matrix that are capable of reacting with other reactive groups on the matrix (also describe in more detail in the above listed patents). The reaction of the groups (such as photo-cyclodimerization) will create local areas of density (higher refractive index). This second mechanism can include photochromism or photorefractives in which case the optical density is changed by a change in the molecular structure of the chromophore. This 2^{nd} type of mechanism has no freely diffusing species, it is considered safer for implantation into an eye.

The third mechanism is loss of a solvent, plasticizer, or other chemical due to a solubility change in the material. Solubility changes can be accomplished by various methods such as changes in pH, changes in hydrophilicity, changes in degree of polymerization, changes in temperature, changes in salinity, etc. For instance, certain azobenzenes can change the hydrophilicity or pH of the local environment upon photo-isomerization. Spiropyrans and pirooxazines can change polarity upon exposure to light; the polarity change can increase or decrease the local hydrophilicity for water. Even simple photodimerization of groups along or pendant to the backbone can cause changes in solubility for solvents. Such changes in hydrophilicity can cause water to preferentially diffuse away from or towards the local region, causing the refractive index to change. This method of refractive index change is particularly useful in the application of the present invention. It is preferable that the change in the concentration of water for an exposed region change by greater than 1 wt %, and preferably greater than 5 wt %. For embodiments whereby a change in shape or a large swelling is desired, changes in the concentration of water greater than 10 wt % are desired, and more preferably changes greater than 50 wt %.

No matter which mechanism is used to change the refractive index or shape, the reversibility may be able to cycle more than once, and in many cases able to cycle more than 2-50 times without significant loss in function or the refractive index contrast. Should contrast degrade with cycling, it is preferred that the refractive index contrast decrease less than 25% over 5 cycles, and more preferred that the refractive index contrast degrade less than 10% over 5 cycles, and most preferred that the refractive index contrast degrade less than 5% over 5 cycles. These degradation rates can also be applied to swelling, volume change, or other characteristics of the material which are affected by the reversibility of the material.

In some embodiments, the cycles will not be degraded by time, such that the write/erase cycle can be performed with varying amounts of time between actions. For example, a writing action is followed by an erase action just minutes afterwards. Another example would be a write action followed by an erase action years later, which then may be followed by a write action only minutes later. Preferably, the chemistry used to change the refractive index is not dependent on the time between actions. In some embodiments, the reversibility of the material may be conserved for more than 1 year, and more preferably 10 years, and most preferably greater than 50 years.

The refractive index contrast that results from a writing step can be greater than 0.005 per, preferably greater than 0.1, and more preferably greater than 0.5 per. Larger changes in refractive index contrast reduce the number of molds needed in making the blanks, since a later photoreaction during a writing step can create the prescription needed. It is understood that the refractive index change that occurs in either the whole lens or parts of the lens may be from the movement of species inside the lens (monomers, water, densification, inert diffusing species, etc.) or it may be from a change in volume or even a change in shape. For instance, if one surface of the lens were to change from hydrophobic to hydrophilic, water would swell into that surface causing the lens to bow. Such a change in shape from swelling is a way to change the focusing power of the lens by changing the curvature of the lens. Similarly, various surface features can be created by selectively swelling or densifying regions of the lens surface. In some embodiments, surface features in combination with refractive index changes within the volume of the lens will be used together. It is also understood that surface features may already be present from a lathing, milling, and/or a molding step and such features may be altered during a writing step.

The formulation may also contain additional components such as plasticizers, co-solvents, mold release compounds, adhesion promoters, dyes, colorants, pigments, antioxidants, UV absorbers, etc. Consider the following example in which a general procedure for making a material of the present invention is described. A material capable of being used as an ophthalmic device is created by first mixing the following components to form a blank: Matrix components in wt %: 50% Desmodur 3900; 7% Ethylene Glycol; 0.5% Dibutyltindilaurate (tin catalyst for urethane cure); and 4% 9-anthracenemethanol (matrix binding site for the write components). Writing components in wt %: 2% 9-anthracenecarbonitrile.

The components may be mixed and placed into a lens shaped mold and cured overnight. Later, the cured material can be removed from the mold, trimmed as needed, and then placed in solvent (water for this particular lens) for a period of time (e.g., 10 minutes) to solvate the lens. Optionally, it may have been inserted into the eye. The material is then ready to record a refractive index pattern such as one that would form a gradient lens or become multifocal. In some embodiments, the writing chemistry may be diffused into the lens after the molding step but before insertion into the eye. This latter technique is useful when the writing components are heat sensitive and the formation of the blank requires heat.

In embodiments whereby it is desired to change the modulus of the lens or portions of the lens, this modulus change can occur either before or after insertion into the eye. Some of the chemistry/mechanisms for altering the modulus after insertion into the eye are flood cure of the lens (irradiation of the whole lens to a light source that causes the lens to be fixed), injection of a catalyst that causes the crosslinking of the IOL material (ex. a change in pH or something like a bismuth based catalyst), injection of a crosslinking agent into the IOL cavity (for example, calcium ions for phosphate polymers), and/or temperature change (potentially provided by the body or infrared light), or any of the photochemistry described in previous sections. The change in modulus can be from polymerization, changes in crosslinking density, solubility changes, or even swelling or deswelling (such as from water). All such modulus increasing reactions may occur within one week, or more preferably less than one hour, and/or more preferably less than five minutes of insertion or of a triggered modulus increasing reaction. It is of particular interest to have an IOL be as small as possible before insertion into the eye, and thus a dried hydrophilic lens can be inserted and then either allowed to hydrate or triggered to hydrate such that it swells with water increasing its size and sometimes its modulus. The change in modulus may be a part of the writing step, and/or may occur before or after insertion into the eye; it may be a separate process using separate chemistry from the writing step. Likewise, the optical patterning may occur before or after the insertion step, and can even occur multiple times during the processing of the lens (before and after insertion into the eye).

It is sometime necessary to remove an installed IOL. In such cases, it is recognized that some of the chemistries/mechanisms of the present invention may also make the removal of a lens more facile. An IOL can be constructed to have photo or chemical degradation groups placed throughout the IOL material to facilitate the degradation of the lens for removal. For instance, if a lens is crosslinked by photodimers, an erasing wavelength can be used to break down the IOL material into smaller and smaller pieces or even have the lens fully dissolve, making removal of the lens material very easy. Or, a group susceptible to photocleavage at far UV wavelengths can be a part of the matrix backbone, and upon the need for removal, either UV light of the correct wavelength is irradiated into the eye, or perhaps through an optical light fiber inserted into the intra ocular lens cavity to degrade the IOL into small enough parts that can be suctioned out or physically removed through a small incision. Additionally, a change in shape of the lens may also facilitate removal, for instance, one surface of the lens could be irradiated such that swelling preferentially occurs on that surface to such an extent as to cause the lens to tightly roll up (like a rolled newspaper).

In previous IOLs, whereby the refractive index is modified post fabrication such as in the present invention, the primary mechanism for this modification is by polymerization of a monomer or oligomer. In such mechanisms, the newly polymerized material is not typically covalently bound to the starting matrix material (unless polymerizable groups are specifically provided on the starting matrix), but is instead entangled or an interpenetrating network is formed of the two polymers (original matrix and the newly formed polymer). In various embodiments, binding to the matrix is the preferred method for refractive index change (in the embodiments that use diffusion of refractive index species). For more description on matrix binding chemistry, see U.S. Pat. No. 8,062,809, which is incorporated herein by reference in its entirety for all purposes.

### Advantages

Existing IOLs correct patient vision by bending rays at the front and back surface of a curved lens. The disclosed method adds a 2D or 3D gradient refractive index to the body of the lens, providing for significantly greater control of the lens performance. Since the crystalline lens of the human eye is a gradient index structure, there is physiological motivation that this degree of control is important.

The ability to customize this gradient structure to the individual patient offers significant potential visual benefits. The human eye operates very far from the theoretical diffraction-limited performance. This has inspired custom eyeglasses and contact lenses to correct the aberrations beyond defocus and astigmatism that are traditional in vision correction today. These “higher order aberration correction” methods have the significant drawback that the artificial lens is not fixed relative to the eye. Eyeglasses are particularly egregious here, but the movement of a contact lens also limits the degree of correction possible. IOLs, on the other hand, are fixed relative to the eye after insertion and thus offer an ideal location for aberration correction. The proposed method should thus enable vision correction beyond 20/20.

A second way this design freedom can be exploited is in the formation of multi-focal IOLs. These compensate for the lack of accommodation by creating several simultaneous focused images at different distances along the optical axis. The visual system rejects the out-of-focus images and concentrates on that nearest to in-focus. However, users complain of glare and poor contrast. Existing multi-focal lenses divide the lens up into annular rings, each of which has a Fresnel lens with different focal lengths. This has a number of disadvantages including diffractive color and scatter off of the sharp transitions between lenses. In contrast, the extra degrees of freedom present in the GRIN structure can be exploited to make multiple foci with very low color dispersion, smooth transitions and better out-of-focus performance. For example, the GRIN lens can be designed to control the position out-of-focus light from other foci to minimize visual interference. Additionally, diffractive GRIN structures can create multiple foci via splitting the light such that there are not distinct regions on the lens that contribute to each focus, minimizing change of lens performance with pupil size. Also, the refractive profile can be designed to create foci with desired shapes including extension of the focus along the axis of the eye to provide extension of the patient depth of focus.

Finally, the use of a final cure to structure the mechanical properties of the lens may be of use in accommodating IOLs. These attach to the ciliary body of the eye in order to change shape and thus focal length, just as the natural crystalline lens does. The ability to tailor the 3D index, modulus or hydration of the lens provides additional design freedom to enable optimal coupling of the ciliary actuation to modify the lens focal length.

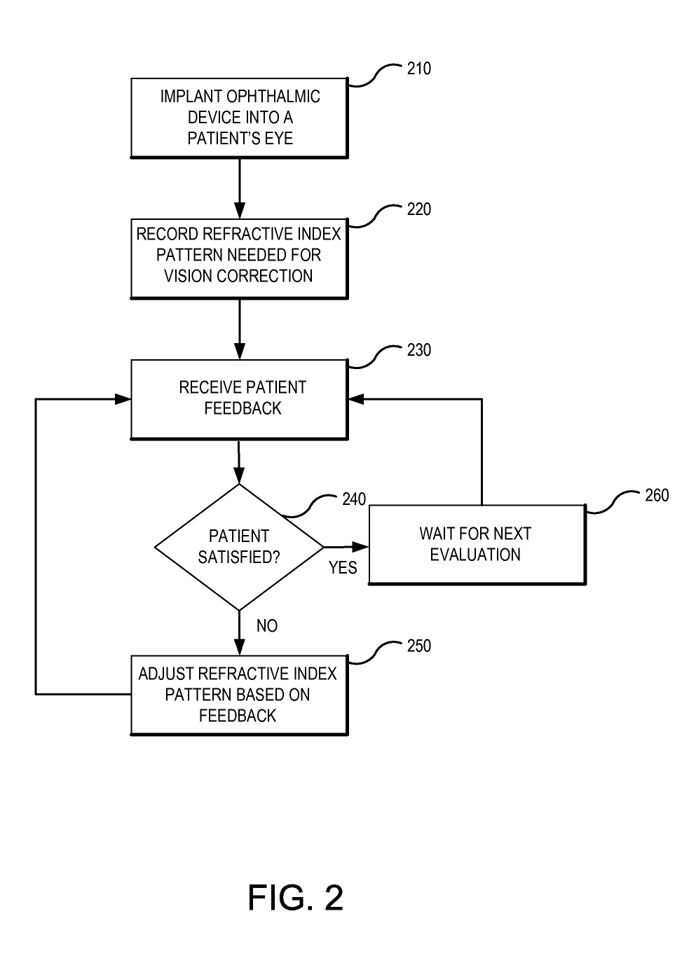

Further, another advantage to the use of rewriteable lenses in the eye is that once the lens is settled into place (or the lens is stabilized in the eye through standard surgical procures such as haptic elements of the IOL), iterative feedback of correction and wavefront correction becomes possible. That is, since rewrite offers ability to non-invasively correct wavefront, one can measure, correct, measure, correct as illustrated in FIG. 2. This should enable non-idealities in the correction mechanism or interactions of patient/IOL aberrations to be fixed to a greater degree. In some embodiments, the writing process is very fast and offers many cycles. As a result, this could potentially replace or augment the traditional “switching of lenses” currently used to determine optimal correction in the office.

As illustrated in FIG. 2, an ophthalmic device may be implanted in to a patient's eye. Recording operation 220 records a refractive index pattern needed (or suspected) for vision correction of a patient. During feedback operation 230, the patient can provide feedback (e.g., orally to the doctor, through a graphical user interface, or other mechanism). This feedback can be used during determination operation 240 to determine if the patient is satisfied or if one or more standards of care have been met. If determination operation 240 determines that the patient is not satisfied or if one or more standards of care have not been met, then determination operation 240 branches to adjustments operation 250 where the refractive index pattern recorded on the rewriteable lens is adjusted. Then, the patient can provide feedback on the current state of the lens during feedback operation 230. If determination operation 240 determines that the patient is satisfied and that the standards of care have been met, then determination operation 240 branches to waiting operation 260 where the process holds until the patient returns for a subsequent evaluation.

A version of the above specific to presbyopia is patient specific presbyopia correction/trial. Multifocal and other techniques for fixed presbyopia correction suffer a high rate of rejection. These reasons apparently go beyond pure optical performance, and include patient preferences, patient lifestyle and possibly details of ocular physiology. A treatment plan that includes the creation of a presbyopia correction followed by patient trial could then adjust the correction based on patient feedback in order to optimize the correction. Examples of customization, assuming a multi-focal approach to correction: Number of foci, placement of foci in pupil, pupil area in focal zone (total irradiance allocated to each focal zone), shape of point-spread function (e.g., strength of side lobes, size of central lobe).

As previously mentioned, the ability to change the patient's lens prescription with time without surgery is very important. The following is just a short list of corrections that can be done over time: 1) Patient aberration changes; 2) Degree of presbyopia; 3) Medically-induced rapid change such as diabetes; and/or 4) Adjustment of correction to optimally work with new, additional corrective lenses such as reading glasses.

Correction of higher-order aberrations that drift with time. “Super vision,” that is correction of weaker aberrations, has the challenge that the eye is dynamic. A Pareto chart of the contribution of aberrations to vision quality has a long tail (that is, many weak contributions). The most significant contributors such as defocus and astigmatism tend to be stable with a time constant of ˜year. However, the higher-order aberrations become less and less stable, making fixed correction of limited value. Thus, the more frequently the patient's correction can be updated, the larger number of aberrations can be corrected. This makes the potential for highly-corrected aberrations more viable with the proposed technology.

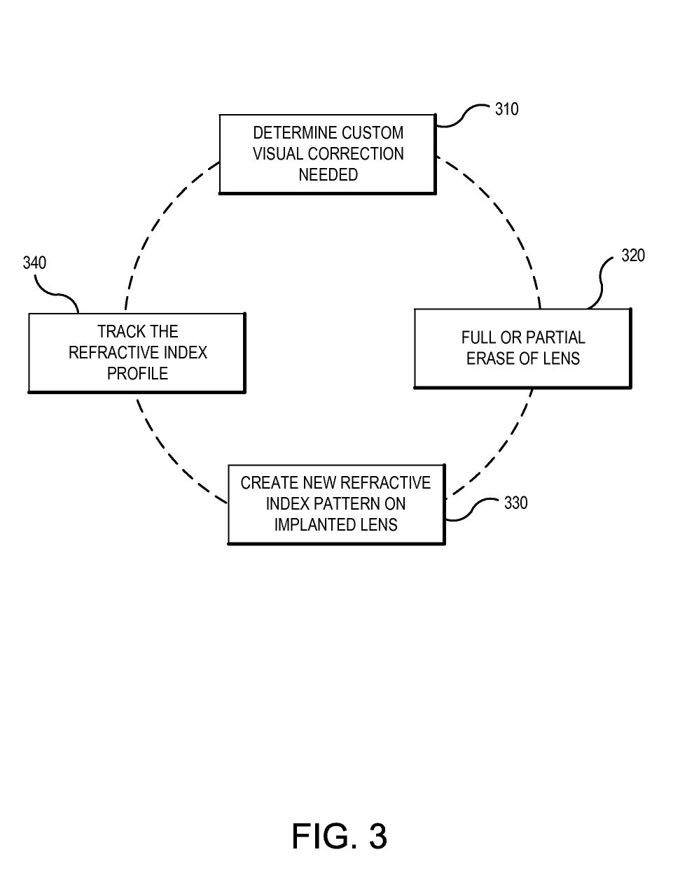

The ability to rewrite the lens does not have to be full erase and full write, partial erase and partial write are also possible. This gives the doctor the ability to gradually change the lens refractive index profile and receive patient feedback to help with the creation of the perfect profile for the patient as illustrated in FIG. 3. The various embodiments illustrated in FIG. 3 provide for the determination of a custom visual correction that may be needed during determination operation 310. During erasure operation 320, a full or partial erase of the lens or ophthalmic device may be completed. A new refractive index pattern may then be recorded on the lens during creation operation 330. During profiling operation 340, the refractive index profile may be track and recorded in a patient's medical record or database.

If the patient's aberration correction history as well as the lens refractive index profile history is stored and tracked, it becomes possible to predict the needed change in the refractive index profile of the lens. In this manner, the patient can have fewer visits to the office since the lens can be written in such a way as to give the patient really good vision that gets better with time and then eventually returns to just good vision at which time the patient would return to the office for another visit. This is in contrast to giving the patient great vision which gradually gets worse with time. Knowing the trend for the patient's aberration(s) as extrapolated from their history allows the doctor to build in a correction prescription that last longer thus extending the time between visits. Also, when correcting higher-order aberrations, the optimal correction would be one that corrects only for those aberration types that are stable in the time period of patient visits. Thus a temporal analysis of the patient aberrations could be used to select a set of aberration terms that, for this particular patient and visit frequency, are sufficiently stable to warrant correction.

Lastly, since this material is able to store refractive index patterns, the material is also capable of storing the patient's aberration history (and the lens history) as data in the lens material itself. This would allow any doctor using standardized equipment or protocols used for this material to read out the patient's ophthalmic history, notes from the previous doctor, even possible medical conditions which may give the patient eye problems. This relieves the patient from having to either remember their eye history (which can be difficult for the elderly) or from the doctor having to request records from another doctor or location (which may no longer be available). And, such data storage can also be used by security services to positively identify individuals beyond a standard retinal scan.

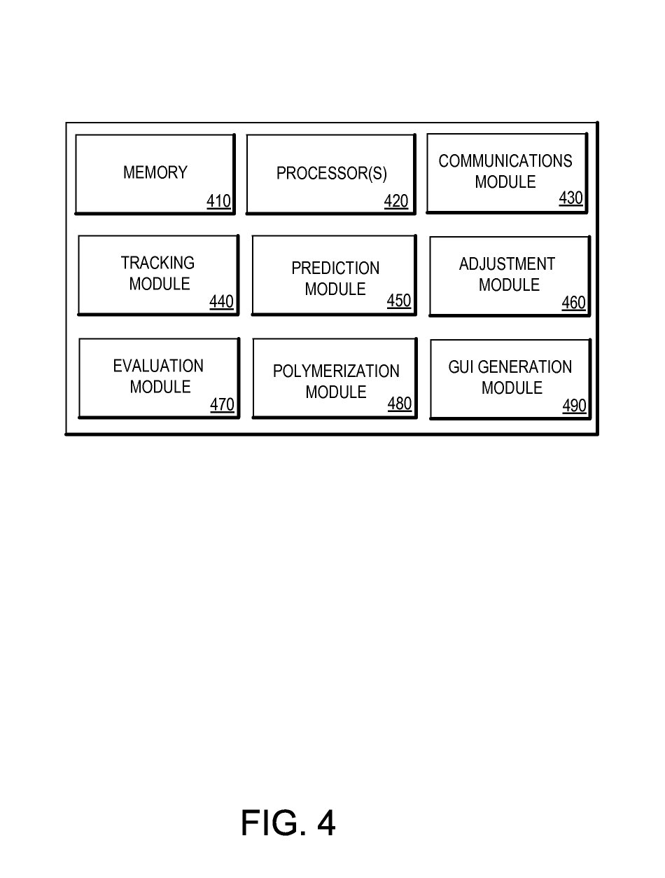

Various embodiments of the present invention may be implemented using a combination of one or more devices, computers, servers, controllers, or engines. These components may use one or more modules as illustrated in FIG. 4. According to the embodiments shown in FIG. 4, devices, computers, servers, controllers, or engines used to implement various embodiments, can include memory 410, one or more processors 420, communications module 430, tracking module 440, prediction module 450, adjustment module 460, evaluation module 470, polymerization module 480, and graphical user interface (GUI) generation module 490. Other embodiments of the present invention may include some, all, or none of these modules and components along with other modules, applications, and/or components. Still yet, some embodiments may incorporate two or more of these modules and components into a single module and/or associate a portion of the functionality of one or more of these modules with a different module.

Memory 410 can be any device, mechanism, or populated data structure used for storing information. In accordance with some embodiments of the present invention, memory 410 can encompass any type of, but is not limited to, volatile memory, nonvolatile memory and dynamic memory. For example, memory 410 can be random access memory, memory storage devices, optical memory devices, media magnetic media, floppy disks, magnetic tapes, hard drives, SDRAM, RDRAM, DDR RAM, erasable programmable read-only memories (EPROMs), electrically erasable programmable read-only memories (EEPROMs), compact disks, DVDs, and/or the like. In accordance with some embodiments, memory 410 may include one or more disk drives, flash drives, one or more databases, one or more tables, one or more files, local cache memories, processor cache memories, relational databases, flat databases, and/or the like. In addition, those of ordinary skill in the art will appreciate many additional devices and techniques for storing information which can be used as memory 410.

Memory 410 may be used to store instructions for running one or more applications or modules on processor(s) 420. For example, memory 410 could be used in one or more embodiments to house all or some of the instructions needed to execute the functionality of communications module 430, tracking module 440, prediction module 450, adjustment module 460, evaluation module 470, polymerization module 480, and/or GUI generation module 490.

In accordance with various embodiments, communications module 430 may be a general-purpose or a special-purpose communications module for interfacing with systems and/or system components capable of writing, erasing, and/or rewriting an index pattern on a lens or other ophthalmic device. Tracking module 440 may be used to track the index pattern needed to correct an individual's eye sight over time. The results may be recorded in one or more databases and the entries may be recorded as differences between patterns, the entire pattern, or in some other format. Prediction module 450 can be used to predict future changes to a patient's vision. In some embodiments, prediction module 450 may access the entries created by tracking module 440. Using these entries along with other data (e.g., age, similar population trends, biological markers, etc.) may be used as input into one or more models which can predict how the patient's vision will evolve over time.

Adjustment module 460 can be used to determine adjustments needed to a recorded index pattern. Adjustment module 460 may send commands to a system for adjusting (e.g., erasing and rerecording) an index pattern on a lens. The adjustments may be determined by evaluation module 470 which evaluates a patient's vision. Polymerization module 480 may be configured to control multi-stage polymerization processes for creating customized lenses. Graphical user interface (GUI) generation module 490 may be used to receive inputs from a doctor or patient. Similarly, GUI generation module 490 may be used to display one or more reports, receive commands for controlling a lens adjustment process, and/or other input/output functionality needed to convey information between a system and a user.

### Exemplary Computer System Overview

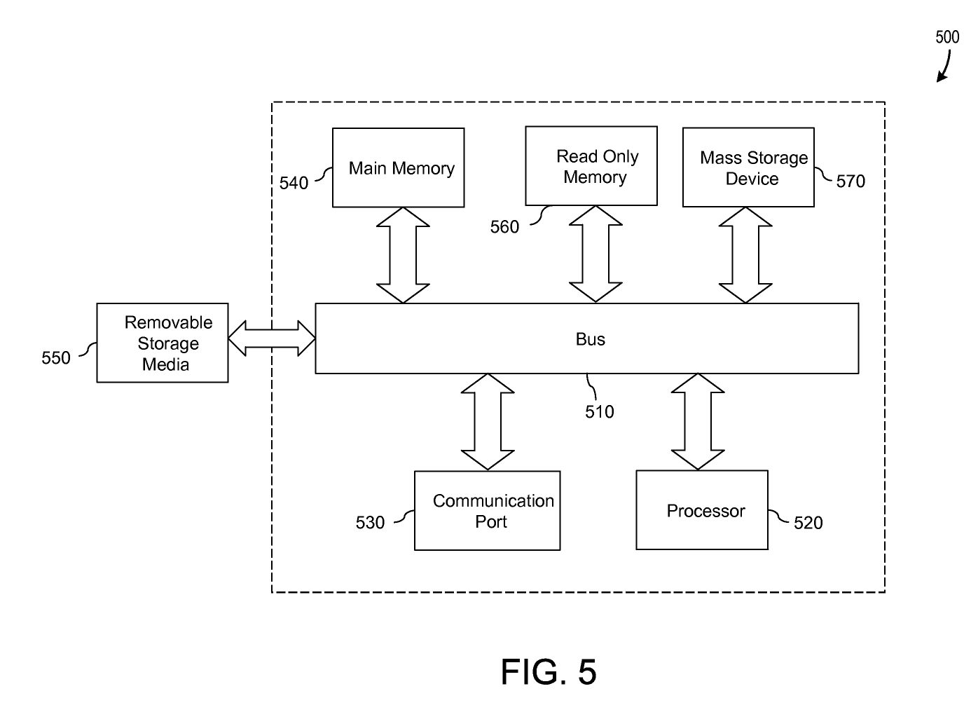

Embodiments of the present invention include various steps and operations, which have been described above. A variety of these steps and operations may be performed by hardware components or may be embodied in machine-executable instructions, which may be used to cause a general-purpose or special-purpose processor programmed with the instructions to perform the steps or cause one or more hardware components to perform the steps. Alternatively, the steps may be performed by a combination of hardware, software, and/or firmware. As such, FIG. 5 is an example of a computer system 500 with which embodiments of the present invention may be utilized. According to the present example, the computer system includes a bus 510, at least one processor 520, at least one communication port 530, a main memory 540, a removable storage media 550, a read only memory 560, and a mass storage 570.

Processor(s) 520 can be any known processor, such as, but not limited to, an Intel® lines of processors, AMD® lines of processors, or Motorola® lines of processors. Communication port(s) 530 can be any of an RS-232 port for use with a modem-based dialup connection, a 10/100 Ethernet port, or a Gigabit port using copper or fiber. Communication port(s) 530 may be chosen depending on a network such a Local Area Network (LAN), Wide Area Network (WAN), or any network to which the computer system 500 connects.

Main memory 540 can be Random Access Memory (RAM), or any other dynamic storage device(s) commonly known in the art. Read only memory 560 can be any static storage device(s) such as Programmable Read Only Memory (PROM) chips for storing static information such as instructions for processor 520.

Mass storage 570 can be used to store information and instructions. For example, hard disks such as the Adaptec® family of SCSI drives, an optical disc, an array of disks such as RAID, such as the Adaptec family of RAID drives, or any other mass storage devices may be used.

Bus 510 communicatively couples processor(s) 520 with the other memory, storage and communication blocks. Bus 510 can be a PCI/PCI-X or SCSI based system bus depending on the storage devices used.

Removable storage media 550 can be any kind of external hard-drives, floppy drives, IOMEGA® Zip Drives, Compact Disc-Read Only Memory (CD-ROM), Compact Disc-Re-Writable (CD-RW), or Digital Video Disk-Read Only Memory (DVD-ROM).

The components described above are meant to exemplify some types of possibilities. In no way should the aforementioned examples limit the scope of the invention, as they are only exemplary embodiments.

Various embodiments of systems and methods for rewriteable devices have been described and set forth. These descriptions and illustrations are not intended to be exhaustive, but rather to highlight some of the benefits and advantages associated with embodiments and features of various embodiments of the present invention. Various modifications and additions can be made to the embodiments discussed without departing from the scope of the technology disclosed. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations and all equivalents thereof.

What is claimed:

1. A method comprising:
recording a prescription on an ophthalmic device composed of a polymeric material that allows the prescription to be erased and rewritten at least once;
determining an adjustment to the prescription; and
recording the adjustment to the prescription on the ophthalmic device.

2. The method of claim 1, further comprising receiving patient feedback regarding the vision of the patient after the recording of the prescription on the rewriteable intraocular lens, and wherein determining an adjustment to the prescription is based, at least in part, on the patient feedback.

3. The method of claim 1, wherein recording the prescription includes overlapping multiple refractive index patterns.

4. The method of claim 1, wherein recording the prescription on the rewriteable ophthalmic device includes fully erasing or partially erasing a previously recorded prescription.

5. The method of claim 1, wherein an average light power used for recording the prescription is less than 100 mw.

6. The method of claim 1, wherein recording the prescription on the rewriteable ophthalmic device includes the creation of multiple foci.

7. The method of claim 1, wherein the ophthalmic device is a intraocular lens and the method further comprises:
tracking movement of an in which the intraocular lens has been inserted; and
recording the adjustment to the prescription on the ophthalmic device includes compensating for the movement so that an optical exposure properly records the adjustment to the prescription.

8. An intraocular lens composed of a material capable of at least one write step and one degradation step wherein a degradation step allows for the facile removal of the intraocular lens.

9. An intraocular lens of claim 9, wherein the degradation step includes a change in shape of the intraocular lens such that the intraocular lens curls up tightly to facilitate removal through a small incision in an eye.

10. The intraocular lens of claim 9, wherein the material include uracil or thymine derivatives.

11. An ophthalmic device using a crosslinked polymeric material capable of recording patterned light as refractive index changes, wherein data is recorded onto the ophthalmic device using changes in refractive index.

12. The ophthalmic device of claim 11, wherein one or more of the following data are recorded on the device: patient's ophthalmic history, patient's prescription history, patient's identification information, device information, recording or erasing parameters.

13. The ophthalmic device of claim 11, wherein the data can be recorded and erased at least once.

14. An ophthalmic device comprising a crosslinked polymeric material with freely diffusing species capable of binding with the crosslinked polymeric material for use in one of the following devices: a phakic lens, an intraocular lens, or a contact lens; wherein the ophthalmic device is shaped by at least one of milling, lathing, molding; and wherein the device is capable of changing its prescription by the use of photochemistry; wherein the photochemistry is reversible.

15. The ophthalmic device of claim 14, wherein the photochemistry chemistry is reversible and uses one or more of the following groups: anthracenes, acenaphthylenes, phenanthrenes, related polyaromatic hydrocarbons, stilbenes, coumarins, maleimides, thymines, uracils.

16. The ophthalmic device of claim 14, wherein the photochemistry chemistry is reversible and uses one or more of the following groups: spiropyrans, pirooxazines, and azobenzenes.

17. The ophthalmic device of claim 14, wherein the photochemistry chemistry is reversible for greater than 5 cycles with less than 25% loss of maximum change in prescription.

18. The ophthalmic device of claim 14, wherein the ophthalmic device is an intraocular lens, and the changes in prescription occur while the device is in the eye.

19. An ophthalmic device using a crosslinked polymeric material with no freely diffusing species contained within the device other than water for use in one of the following devices: a phakic lens, a psuedophakic lens, an intraocular lens, or a contact lens; wherein the ophthalmic device is shaped by at least one of milling, lathing, molding; wherein the ophthalmic device is capable of changing its prescription by the use of photochemistry; and wherein the photochemistry changes the concentration of water over a portion or over the whole of the device.

20. The ophthalmic device of claim 19, wherein a refractive index contrast obtained during a writing step is greater than 0.005 between an exposed region and an unexposed region.

21. The ophthalmic device of claim 19, wherein the ophthalmic device is capable of water concentration changes greater than 5 wt % upon exposure to light to which the material is sensitive.

22. The ophthalmic device of claim 21, wherein the chemistry causing the change in water concentration is from photocyclodimerization of the matrix with itself, using one or more of the following groups: at least one of the following groups: anthracenes, acenaphthylenes, phenanthrenes, related polyaromatic hydrocarbons, stilbenes, coumarins, maleimides, thymines, uracils.

23. The ophthalmic device of claim 21, wherein the chemistry causing the change in water concentration is from at least one of the following: spiropyrans, pirooxazines, azobenzenes.

24. The ophthalmic device of claim 21, wherein the chemistry causing the change in water concentration is photoreversible.

25. The ophthalmic device of claim 24, wherein the change in water concentration is reversible for greater than five cycles with less than 25% loss of maximum change in concentration.

26. The ophthalmic device of claim 25, wherein the ophthalmic device is an intraocular lens, and the changes in water concentration occur during a photoreaction while the device is in the eye.

27. An ophthalmic device that uses a crosslinked polymeric material capable of recording erasable refractive index patterns for more than two write-erase cycles with less than 20% degradation to the maximum refractive index contrast of which the material is capable.

28. The ophthalmic device of claim 27, wherein the ophthalmic device is optimized for use with other eyewear.

29. The ophthalmic device of claim 28, wherein the other eyewear includes traditional glasses or an exterior head mounted display.

30. An ophthalmic device that has surface features that were molded, milled, or lathed onto the ophthalmic device and the ophthalmic device comprises of a material capable of recording light patterns as refractive index patterns in the volume of the material.

31. The ophthalmic device of claim 30, wherein the refractive index patterns are rewriteable.
